(19) United States
(12) Patent Application Publication
(10) Pub. No.: US 2026/0338792 A1
(43) Pub. Date: Oct. 29, 2026

(54) HIGH EFFICIENCY ANTIBODIES FOR CHROMATIN TARGETS

(71) Applicant: EPICYPHER, INC., Durham, NC (US)

(72) Inventors: Martis W. Cowles, Chapel Hill, NC (US); Andrea L. Johnstone, Apex, NC (US); Danielle Maryanski, Durham, NC (US); Keli Rodriguez, Haw River, NC (US); Matthew Marunde, Carrboro, NC (US); Bryan Venters, Apex, NC (US); Zu-Wen Sun, Brentwood, TN (US); Michael-Christopher Keogh, Cambridge, MA (US)

(21) Appl. No.: 19/117,757

(22) PCT Filed: Oct. 12, 2023
(86) PCT No.: PCT/US2023/076682

§ 371 Date: Apr. 2, 2025

Related U.S. Application Data
(60) Provisional application No. 63/379,232, filed on Oct. 12, 2022.

(51) Int. Cl. G01N 33/68; C07K 16/44

(52) U.S. Cl. CPC G01N 33/6875; C07K 16/44; G01N 33/6854; G01N 2440/00

(57) ABSTRACT

Disclosed herein are methods to develop, assay and identify high efficiency antibodies and high efficiency antibody fragments that target histone post-translation modifications or DNA modifications. Improved genomic mapping assays using the identified high efficiency antibodies and/or antibody fragments are also disclosed. High efficiency antibodies and antibody fragments developed and optimized by the disclosed methods are also provided.

### STATEMENT OF PRIORITY

This application claims the benefit of U.S. Provisional Application Ser. No. 63/379,232, filed Oct. 12, 2022, the entire contents of which are incorporated by reference herein.

### FIELD OF INVENTION

The invention relates to a novel method to develop and/or identify high efficiency antibodies that target chromatin. The invention further relates to the use of these improved high efficiency antibodies in assays for chromatin analysis.

### BACKGROUND

Low input, Single Cell (SC) and spatial analyses are revolutionizing the biological sciences, facilitating a new era in understanding of cellular dynamics and variation (Stuart and Satija 2019). Low input bulk assays are essential to analyze limited samples, such as sorted cell populations or clinical samples. For example, many crucial target populations yield as few as 5K cells per mouse (e.g., positively selected germinal center B cells). Thus, ultra-sensitive assays are required to generate reliable, high-quality data from these precious samples. In contrast, SC analysis yields specific data for individual cells of a heterogeneous input pool that might otherwise be lost or masked in aggregate analyses. Indeed, SC resolution provides the power to identify and characterize biologically relevant details between cells, including rare subpopulations (Vegh and Haniffa 2018), chemoresistant tumor cells (Kim, Gao et al. 2018), myeloid cell states (Zilionis, Engblom et al. 2019), and novel cell types related to disease (Plasschaert, Zilionis et al. 2018). Spatial assays are enabling analysis of one or more cells directly in intact tissues (Deng, et al., Science, 2022; Williams, Lee, and Asatsuma et al., 2022; Vandereyken, 2023; Moffitt, et al., 2022). These assays have the potential of providing similar resolution as SC assays while retaining spatial context information.

Low input approaches (including SC and spatial) for chromatin mapping are beginning to emerge but have limitations. Compared to transcriptomics (e.g., RNA-seq), epigenomics is more technically challenging due to the presence of only two copies of the genetic material per cell, requiring methods to be exquisitely sensitive, robust, and reliable. As a result, few chromatin-based approaches have been successfully adapted for low input, SC, or spatial applications, most notably bisulfite sequencing to assess DNA methylation (Smallwood, Lee et al. 2014; Ferreyra Vega, Wenger, Kling et al., 2022; Ahn, et al. 2021) and ATAC-seq for chromatin accessibility (Buenrostro, Giresi et al. 2013; Deng, et al., Nature 2022; Fang, Preissl, Li et al., 2021). Unfortunately, these assays cannot differentiate histone post-translational modifications (PTMs) that label various chromatin features (e.g., promoters, enhancers, etc.) or transcription factors that control gene regulation, thereby limiting their utility. It is important to note that while ChIP-seq (commonly used for histone PTM mapping) has recently been adapted for SC (scChIP/Drop-ChIP (Rotem, Ram et al. 2015, Grosselin, Durand et al. 2019)), these methods exhibit high background and sequencing costs and are challenging to scale due to challenges in sample processing (e.g., cell lysis and chromatin fragmentation via mechanical or enzymatic means). New ‘chromatin tethering’ methods affix enzymes to specific genomic regions, resulting in labeling/release, and selective analysis of target material (e.g., DamID, ChIC, CUT&RUN, and CUT&Tag). CUT&RUN/CUT&Tag are poised to become the leading enzyme-mediated methods (Skene and Henikoff 2017, Skene, Henikoff et al. 2018), greatly expanding upon Chromatin ImmunoCleavage (ChIC; (Schmid, Durussel et al. 2004)).

CUT&RUN and ChIC use a factor-specific antibody to tether a fusion protein of protein A/protein G and micrococcal nuclease (pAG-MNase) to genomic binding sites in intact cells, which is then activated by the addition of calcium to cleave DNA. pAG-MNase provides a cleavage tethering system for antibodies to any PTM, transcription factor, or chromatin protein of interest. The CUT&RUN protocol can be further streamlined by using a solid support to adhere cells (or nuclei) to lectin-coated magnetic beads, generating reliable genomic mapping data using as few as 100 cells and 3 million reads. These advances simplify processing, dramatically increase sample recovery, and enable protocol automation.

Similar to CUT&RUN, CUT&Tag uses antibodies to bind chromatin proteins in situ, and then tethers a protein A/protein G and hyperactive Tn5 transposase (pAG-Tn5) fusion to these sites. Upon controlled activation, the Tn5 selectively fragments and integrates adapter sequences at the genomic sites. The tagged target DNA is then amplified and sequenced, thereby bypassing several library preparation steps, saving time (total workflow time of 1-2 days) and eliminating a source of experimental bias. The CUT&Tag approach has remarkable signal-to-noise (S/N), generating high quality genomic mapping data using as few as 10 cells (Skene, Henikoff et al. 2018, Meers, Bryson et al. 2019) and 1-3 million (M) reads (vs. >50K cells and >30M reads for ChIP-seq) for select highly abundant histone PTM targets. Groups have successfully developed SC CUT&Tag first using established SC platforms, including the ICELL8 platform (Kaya-Okur, Wu et al. 2019) and the Chromium platform from 10× Genomics (Wu, Furlan et al. 2021). Of note, other immunotethering-based approaches have also been developed for genomic mapping in SCs (e.g., scChIC-seq (Ku, Nakamura et al. 2019), CoBATCH (Wang, Xiong et al. 2019), scCUT&RUN (Hainer, Boskovic et al. 2019)). Further, multiomic CUT&Tag (e.g., Paired-Tag (Zhu, Zhang et al. 2021), scCUT&Tag-Pro (Zhang, Srivastava et al. 2021)) and spatial-CUT&Tag (Deng, Bartosovic et al. 2022) assays have recently been established, demonstrating the broad utility for chromatin tethering methods to advance the field of low input, spatial, and SC chromatin profiling.

Despite S/N improvements offered by CUT&RUN and CUT&Tag, low input and SC applications still present a formidable challenge. Indeed, in order to generate high-quality data, low input and SC assays require antibodies to exhibit exquisite on-target epitope binding (i.e., efficiency) with minimal off-target binding (i.e., specificity). As such, antibody efficiency in particular becomes paramount in low input/SC studies, where it directly impacts recovery and data output (i.e., unique reads per sample or SC). Of note, current low input/SC histone PTM mapping studies have mainly focused on a few very robust and prevalent histone PTM targets (e.g., H3K27me3, a marker of heterochromatin and found on 20-30% of nucleosomes (Peach, Rudomin et al. 2012)).

Thus, improved reagents are greatly needed to not only improve signal for well-studied robust targets, but also enable access to other high-value PTMs or transcription factors that are labile or less prevalent (e.g. H3K4me3, a marker of active promoters, is found on <0.5% of nucleosomes (Peach, Rudomin et al. 2012)). Antibodies with improved target efficiency will be of broad use for chromatin assays that use low cell numbers or even single cells.

### INVENTION SUMMARY

The present invention is based, in part, on development of a novel method of identifying antibodies that exhibit a >5-10× increase in nucleosome capture efficiency versus current best-in-class antibodies that were selected using histone peptides. Significantly, the identified antibodies can be used for improved signal to noise in a low input genomic mapping assay.

In an aspect, a method for developing a high efficiency antibody or antibody fragment that targets a histone PTM or DNA modification is provided, comprising measuring binding specificity of antibody or antibody fragment candidate clones to a recombinant nucleosome substrate comprising the PTM or DNA modification, thereby identifying a first set of antibodies targeting the histone PTM or DNA modification; and validating antibodies or antibody fragments from the first set of antibodies by performing a genomic mapping assay, wherein the first set of antibodies are evaluated for both binding specificity and efficiency, thereby identifying the high efficiency antibody or antibody fragment that targets the histone PTM or DNA modification. In some embodiments, the method comprises identifying more than one high efficiency antibody or antibody fragment, wherein each high efficiency antibody or antibody fragment targets a different histone PTM or DNA modification and the recombinant nucleosome substrate comprises each of the PTMs or DNA modification. In one embodiment, the validating step is performed with recombinant nucleosomes substrates carrying the histone PTMs and/or DNA modifications.

In another aspect, a method for developing a high efficiency antibody or antibody fragment that targets a chromatin binding protein is provided, comprising measuring binding specificity of antibody or antibody fragment candidate clones to a nucleosome substrate comprising an engineered epitope, the epitope found on the target chromatin binding protein, thereby identifying a first set of antibodies targeting the chromatin binding protein; and validating antibodies or antibody fragments from the first set of antibodies by performing a genomic mapping assay, wherein the first set of antibodies are evaluated for both binding specificity and efficiency, thereby identifying the high efficiency antibody or antibody fragment that targets the chromatin binding protein.

In some embodiments, measuring binding specificity comprises performing an enzyme-linked immunosorbent assay (ELISA) or performing a multiplex assay, optionally a Luminex assay.

In some embodiments, the genomic mapping assay is ChIP-seq, CUT&RUN, or CUT&Tag. In some embodiments, the genomic mapping assay comprises nucleosome spike-in controls that comprise on-target and off-target epitopes. In some embodiments, the genomic mapping assay comprises evaluating antibody or antibody fragment binding efficiency at a range of cell inputs. In some embodiments, the identified high efficiency antibody or antibody fragment generates substantially similar data at 500,000 cells and at fewer than 100,000, 50,000 25,000 10,000, 5000, 1000, 100, or 10 cells. In some embodiments, data quality is determined using Fragment of Reads in Peaks (FRiP) score or replicate analysis (i.e., Pearson correlation).

In some embodiments, the antibody or antibody fragment is a polyclonal antibody or antibody fragment, monoclonal antibody or antibody fragment, recombinant polyclonal antibody or antibody fragment, recombinant monoclonal antibody or antibody fragment, or nanobody.

In some embodiments, each step of the methods is performed using recombinant nucleosome substrates. In some embodiments, at least one step is performed using recombinant nucleosome substrates.

In another aspect, a high efficiency antibody or antibody fragment identified by the methods described herein is also provided.

In a further aspect, a method of performing a genomic assay is provided, comprising using the high efficiency antibody or antibody fragment of the present invention. In an embodiment, the genomic mapping assay is ChIP-seq, CUT&RUN, or CUT&Tag. In some embodiments, the assay is performed using fewer than 100,000, 50,000, 25,000, 10,000, 1,000, 100, or 10 cells. In some embodiments, the assay is performed using a single cell.

These and other aspects of the invention are set forth in more detail in the description of the invention below.

### BRIEF DESCRIPTION OF THE DRAWINGS

FIG. 1. Schematic of example embodiment dNuc-based antibody development pipeline.

FIGS. 2A-2D. Testing of 24 lysine methylation antibodies in (A) SNAP-ChIP and (B) Luminex-based workflows, using the lysine methylation status (K-MetStat) dNuc panel. (C, D) Heatmaps for antibody performance. Rows represent tested antibodies and columns represent dNuc standards. Binding is represented by a gradient of orange (low specificity) to dark blue (high specificity). Testing against SNAP spike-ins also generates antibody efficiency scores (C, light to dark purple).

FIG. 3. Heatmap of H3K4me3 antibodies with high (71%; right) or low (10%; left) efficiency scores flanking annotated transcription start sites (TSSs; +/−3 kb).

FIGS. 4A-4C. Example embodiment of screening of H3K4me3 recombinant polyclonal antibody candidates. (A) Round 1 screening with peptides or dNucs shows poor correlation between candidates that pass (above grey line) both metrics (black stars pass peptide, fail dNuc; gray stars pass peptide and dNuc). (B) Round 2 example dCypher-Luminex assay screening from selected clones in A (arrows) revealed passing peptide screening was a poor indicator of performance in dCypher-Luminex. (C) Antibodies that perform well in dCypher-Luminex exhibit high efficiency via example Round 3 SNAP-ChIP.

FIGS. 5A-5B. Specificity and efficiency profiles for H3K4me1 (A) and H3K4me3 (B) example embodiment antibodies developed according to methods of the invention.

FIGS. 6A-6B. CUT&RUN analysis for H3K4me1 (A) and H3K4me3 (B) in K562 cells. IGV tracks scaled by input (i.e., 100K, 30K, 10K). At high inputs, both antibodies produced adequate data, whereas data falls apart with lower cell input with Low Efficiency (LE) antibody; see signal disappearing in LE antibody tracks. Signal is maintained even at low cell inputs with example High Efficiency (HE) antibody identified according to exemplary method.

FIGS. 7A-7C. Overview of SNAP platforms (A) and efficiency calculations (B). SNAP-CUT&Tag reproduces SNAP-ChIP efficiency for H3K4me3 HE and LE antibodies (C).

FIGS. 8A-8E. The use of recombinant nucleosomes during antibody development according to an exemplary embodiment identifies clones that would have been missed using histone peptide substrates. (A) Table showing representative antibody clone selection results using various assays. Luminex-peptide results is a multiplex assay that uses modified histone peptides conjugated to Luminex beads. Luminex dNuc results refers to a multiplex assay that uses dNucs conjugated to Luminex beads. Spike-in Nucleosome refers to CUT&RUN assay performed using DNA-barcoded nucleosomes to determine antibody specificity. Cell Titration assays were CUT&RUN assays performed using a range of different cell inputs. Top Clone refers to the best candidate selected based on specificity and efficiency metrics. (B) Representative Luminex dNuc results. (C-D) Example of two clones that pass or fail specificity testing using Luminex dNuc assay. (E) Representative IGV tracks of cell titration experiment using Clone 3, demonstrating high target binding efficiency.

FIG. 9. Comparison of example high efficiency H3K36me2 antibody (Clone 3 from FIG. 8A-8B) against the best-in-class commercial H3K36me2 antibody (Active Motif; Cat #61019) in CUT&RUN assays using 500K or 50K cells.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for cloning genes, amplifying, and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties (e.g., remodeling activity) of at least about 50% or more as compared to the activity of a polypeptide consisting of the recited sequence.

As used herein, the term “polypeptide” encompasses both peptides and proteins, unless indicated otherwise.

The terms “polynucleotide,” “nucleic acid,” “nucleic acid molecule,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, genomic DNA, chimeras of RNA and DNA, isolated DNA of any sequence, isolated RNA of any sequence, synthetic DNA of any sequence (e.g., chemically synthesized), synthetic RNA of any sequence (e.g., chemically synthesized), nucleic acid probes and primers. A polynucleotide can comprise modified nucleotides, such as methylated nucleotides and nucleotide analogs or derivatives (e.g., inosine or phosphorothioate nucleotides). Such nucleotides can be used, for example, to prepare nucleic acid molecules that have altered base-pairing abilities or increased resistance to nucleases.

As used herein, a “functional” polypeptide or “functional fragment” is one that substantially retains at least one biological activity normally associated with that polypeptide (e.g., wild-type protein or fragment thereof). In particular embodiments, the “functional” polypeptide or “functional fragment” substantially retains all of the activities possessed by the unmodified polypeptide (e.g., wild-type protein or fragment thereof). By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as chromatin binding activity can be measured using assays that are well known in the art and as described herein.

The term “fragment,” as applied to a peptide, will be understood to mean an amino acid sequence of reduced length relative to a reference peptide (e.g., wild-type protein) or amino acid sequence and comprising, consisting essentially of, and/or consisting of an amino acid sequence of contiguous amino acids identical to the reference peptide or amino acid sequence. Such a peptide fragment according to the invention may be, where appropriate, included in a larger polypeptide of which it is a constituent. In some embodiments, such fragments can comprise, consist essentially of, and/or consist of peptides having a length of at least about 5, 10, 15, 20, 25, 30, 35, 46. 50, 55, 60, 65, 70, 75, 80, 85, 90, 95 or 100 or more consecutive amino acids of a peptide or amino acid sequence according to the invention.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof refers to bringing two or more substances in sufficiently close proximity to each other for one to exert a biological effect on the other.

As used herein, a high efficiency or antibody fragment exhibits high on-target epitope binding (i.e., efficiency) with minimal off-target binding (i.e., specificity). In some embodiments, a high efficiency antibody can be measured relative to commercially available antibodies. In some embodiments, a high efficiency antibody exhibits an increase in nucleosome capture efficiency vs. current best-in-class antibodies or commercial antibodies that were selected using peptides. In some embodiments, the increase in nucleosome capture efficiency is 2 times. 3 time, 4, times, 5, times, 6 times, 7 times, 8 times, 9, times 10 times or more versus a best-in class antibody or commercial antibody that was selected using a peptide approach (e.g., histone peptides). In some embodiments, a high efficiency antibody or antibody fragment can generate substantially similar data in a genomic mapping assay with an input of 500,000 cells and at fewer than 100,000, 50,000, 25,000 10,000, 5000, 1000, 100, or 10 cells. Data quality can be assessed using, for example, Fragment of Reads in Peaks (FRiP) score or replicate analysis (i.e., Pearson correlation).

The present invention relates to a novel antibody screening pipeline for identifying chromatin targeting antibodies that exhibit high binding efficiency in a nucleosomal context, and chromatin targeting antibodies identified by the screening pipeline. The invention further relates to methods that use said chromatin targeting antibodies as well as assay kits that include the reagents needed to perform various assays.

As described above, epigenomic mapping of limited samples or single cells (SCs) is required to study the epigenetic landscape of rare and heterogenous cell populations. However, mapping chromatin elements such as histone PTMs, DNA modifications, or chromatin binding proteins using low cell numbers or at SC resolution presents a unique challenge, as these assays generate sparse data and require antibodies to exhibit high on-target epitope binding (i.e., efficiency) with minimal off-target binding (i.e., specificity). Most antibody development pipelines use modified histone peptides to assess on-target recognition and specificity. However, antibody binding to histone peptides does not correlate with specificity in vivo, such as in genomic mapping assays (Shah, Grzybowski et al. 2018). Indeed, histone peptides fail to account for the complex interactions that exist on chromatin in vivo as well as various assays, immunoblot assays require protein denaturing whereas genomic mapping assays often target chromatin inside intact cells (e.g., ChIC, CUT&RUN and/or CUT&Tag) or chromatin that has been isolated from the cell (or nuclei; e.g., ChIP-seq).

The present methods describe an improved antibody development pipeline that identifies high efficiency antibodies and antibody fragments that exhibit high on-target epitope binding (i.e., efficiency) with minimal off-target binding (i.e., specificity). In some embodiments, the method identifies ultra-efficient histone PTM antibodies for ultra-low input, SC, or spatial assay applications. In some embodiments, the methods of the present invention use recombinant modified nucleosome technology during one or more steps of the method. In some embodiments, use of recombinant modified nucleosomes occur at each (or select) steps of antibody selection in the method enables the identification of highly specific antibodies that exhibit a >5-10× increase in binding efficiency vs. current antibodies developed using peptide-based approaches. The methods described herein enable the development of first-in-class “SC-grade”antibodies, which will be used to enable novel low input and SC epigenetic assays for research, drug development, and biomarker development.

Nucleosomes are the repeating unit of chromatin, comprised of DNA wrapped around a histone octamer (containing two copies each of the histone proteins H2A, H2B, H3, and H4). A central aspect of the present strategy is the use of recombinant nucleosomes as substrates for antibody selection. By incorporating recombinant nucleosomes as part of the antibody clone selection process in the present method, Applicant has reliably identified highly specific antibodies that also exhibit a >5-10× increase in binding efficiency vs. those developed using histone peptides (see, e.g., Example 1). Importantly, by performing head-to-head antibody screening tests using either histone peptides or recombinant nucleosomes, disclosed herein is the discovery that nucleosomes (vs histone peptides) are capable of identifying unique clones that exhibit high binding affinity in the nucleosome context and would have been otherwise missed by state-of-the-art histone peptide-based screening approaches.

In some embodiments, the present invention relates to methods for identifying one or more antibodies that target one or more histone PTMs. In the present invention, recombinant nucleosomes are used to characterize the binding specificity and affinity of candidate clones derived using any antibody production method known in the art, including, for example, polyclonal (Leenaars and Hendriksen 2005), monoclonal (Leenaars and Hendriksen 2005), recombinant (Kunert and Reinhart 2016), phage display (Alfaleh, Alsaab et al. 2020), and single domain antibodies (Harmsen and De Haard 2007).

In some embodiments, a method for developing a high efficiency (HE) antibody that targets a histone post-translational modification (PTM) is provided, comprising: profiling binding specificity of antibody candidate clones to recombinant nucleosome substrates comprising the PTMs, thereby identifying a first set of antibodies targeting the histone PTM; and validating antibodies from the first set of antibodies by performing a genomic mapping assay, wherein the first set of antibodies are evaluated for both binding specificity and efficiency, thereby identifying the high efficiency antibody that targets the histone PTM.

Methods for screening a detection reagent (e.g., an antibody or antibody fragment) for a chromatin element for desired characteristics, e.g., specificity and/or efficiency, are provided, comprising: providing a panel of recombinant nucleosomes, each nucleosome comprising one or more chromatin elements recognized by the one or more binding domains present in the detection reagent and/or one or more chromatin elements not recognized by the one or more binding domains present in the detection reagent, thereby providing both on-target and off-target recombinant nucleosomes; and performing a genomic assay with the panel of recombinant nucleosomes to identify the binding specificity and/or efficiency of the detection reagent. In one embodiment, the detection reagent is a recombinant fusion protein comprised of one or more binding domains that recognize one or more of the chromatin elements of at least one of the nucleosomes in the panel, and a label. The genomic assay can comprise a genomic mapping assay such as CUT&RUN or a binding assay, such as a proximity bead-based assay such as dCypher® (see, Marunde, et al., (2022). The dCypher Approach to Interrogate Chromatin Reader Activity Against Posttranslational Modification-Defined Histone Peptides and Nucleosomes. In: Horsfield, J., Marsman, J. (eds) Chromatin. Methods in Molecular Biology, vol 2458. Humana, New York, NY; doi: 10.1007/978-1-0716-2140-O_13, incorporated herein by reference in its entirety) or a bead-based multiplex assay such as Luminex®-dCypher® assay.

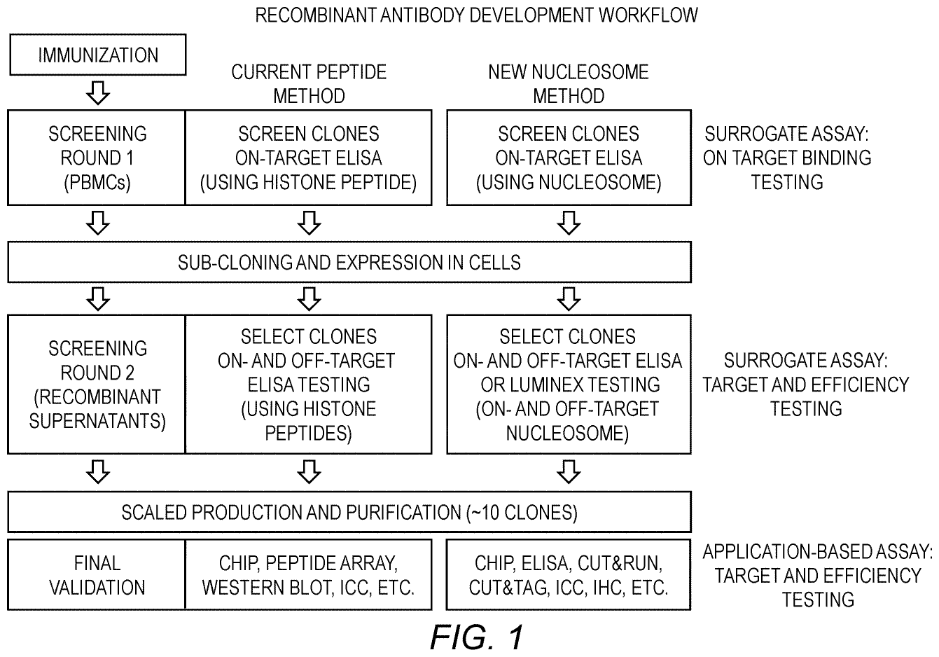

An example method for developing antibodies using a recombinant antibody development workflow using nucleosome substrates of the present invention include the workflow depicted in FIG. 1 along with a head-to-head comparison with current peptide-based screening approaches. In this example, i) rabbits are immunized with a peptide containing the target epitope, ii) rabbit B-cells are isolated from PBMCs and antibody or antibody fragment candidate clones are profiled for antigen reactivity using one or more recombinant nucleosome substrates comprising the PTM, thereby identifying a sets of antibodies that targeting a specific histone PTM in a nucleosome context (Screening Round 1 in FIG. 1), iii) B-cells that contain clones with high binding specificity/efficiency are then subcloned and transfected into mammalian cells, iv) supernatant from transfected cells are then profiled for antibody binding and specificity using a collection of nucleosomes that contain both on and off target PTMs (Screening Round 2 in FIG. 1), and v) selected antibodies are then purified for final validation using an application based assay (Final Validation in FIG. 1).

An example method for developing the antibodies or antibody fragments can comprise steps that include profiling binding specificity of antibody or antibody fragment candidate clones to a recombinant nucleosome substrate comprising the PTM, thereby identifying a first set of antibodies targeting the histone PTM; and validating antibodies or antibody fragments from the first set of antibodies by performing a genomic mapping assay, wherein the first set of antibodies are evaluated for both binding specificity and efficiency, thereby identifying the high efficiency antibody or antibody fragment that targets the histone PTM. In some embodiments, the method comprises identifying more than one antibody or antibody fragment, wherein each antibody or antibody fragment targets a different histone PTM and the recombinant nucleosome substrate comprises each of the PTMs. In some embodiments, the validating step is performed with recombinant nucleosomes substrates carrying the histone PTMs.

In some embodiments, a method for developing a high efficiency antibody or antibody fragment that targets a chromatin binding protein is provided, comprising measuring binding specificity of antibody or antibody fragment candidate clones to a nucleosome substrate comprising an engineered epitope, the epitope found on the target chromatin binding protein, thereby identifying a first set of antibodies targeting the chromatin binding protein; and validating antibodies or antibody fragments from the first set of antibodies or antibody fragments by performing a genomic mapping assay, wherein the first set of antibodies or antibody fragments are evaluated for both binding specificity and efficiency, thereby identifying the high efficiency antibody or antibody fragment that targets the chromatin binding protein.

In some embodiments, the measuring step comprises performing enzyme-linked immunosorbent assay (ELISA). In some embodiments, the measuring step comprises performing a multiplex assay, optionally a Luminex assay. In some embodiments, the step of validating the first set of antibodies or antibody fragments comprises performing a genomic mapping assay. In some embodiments, the application-based assay is a genomic mapping assay (e.g., ChIP-seq, CUT&RUN, or CUT&Tag).

In some embodiments, the initial antibody/antibody fragment screening steps are performed using peptides (or recombinant proteins; e.g., histone or transcription factor) and the latter steps are performed using nucleosomes. In some embodiments, the initial antibody/antibody fragment screening steps are performed using nucleosomes and the latter steps are performed using peptides. In some embodiments, the use of peptides and nucleosomes can be used for alternating steps throughout the antibody production pipeline. In some embodiments, all screening steps are performed using nucleosome substrates. In some embodiments, each method step is performed using recombinant nucleosome substrates. In some embodiments, at least one step is performed using recombinant nucleosome substrates. In some embodiments, the method comprises the step of measuring binding specificity of antibody or antibody fragment candidate clones to a recombinant nucleosome substrate comprising a PTM. In some embodiments, validating antibodies or antibody fragments from the first set of antibodies can comprise performing a genomic mapping assay, wherein the genomic mapping assay comprises recombinant nucleosome substrates.

In the present invention, any type of nucleosome-based assay can be used for antibody clone characterization and/or screening, including the steps of measuring binding specificity of antibody or antibody fragment candidate clones and validating antibodies or antibody fragments. Example nucleosome-based assays include ELISA, Luminex, and/or immunoblot. These assays are useful to determine antibody or antibody fragment binding specificity (i.e., determine the binding preference of one histone and/or DNA modification versus another). In some embodiments, the front-line screen is performed using a single modified nucleosome and unmodified nucleosome to determine on-target binding in a nucleosomal context. In some embodiments, subsequent assays can be performed using a range of different modified nucleosomes to determine antibody or antibody fragment specificity, such as a Luminex-based multiplex assay wherein each fluorescent bead is coupled to a different modified nucleosome. In some embodiments, antibody or antibody fragment efficiency can be determined using any method known in the art. For example, in the case of an ELISA and/or Luminex multiplex assay, antibody binding efficiency can be determined by max signal, whereas the clones with the highest maximum signal are predicted to exhibit the highest binding affinity. However, binding efficiency is best determined ‘in application’ as antibody performance in surrogate assays sometimes does not replicate in the target assay. For example, the binding efficiency in an ELISA and/or Luminex assay may not directly transfer to high binding affinity in genomic mapping studies, as the assay wash conditions, biophysical workflow, and/or readout may be different and impact antibody performance. In some embodiments, antibody binding efficiency is determined in the desired final application, such as ChIP-seq, CUT&RUN, or CUT&Tag. In these instances, it can be useful to use DNA-barcoded spike-in nucleosomes to determine antibody specificity and/or efficiency as previously described (see, e.g., U.S. Pat. Nos. 10,732,185 and 10,787,697). Antibody efficiency can be determined using DNA-barcoded spike-in nucleosomes by comparing the amount of actual nucleosome capture vs. theoretical; see Example 3 for details of an exemplary method to determine antibody or antibody fragment efficiency using immunotethering strategies, such as CUT&RUN/CUT&Tag. In some embodiments, the DNA-barcoded spike-in nucleosomes contain a histone PTM that corresponds to a specific barcode sequence. In some embodiments, the DNA-barcoded spike-in nucleosomes contain a binding epitope for a chromatin binding protein (e.g., transcription factor, chromatin binding protein, remodeling enzyme, etc.) or epitope tag (e.g., FLAG, 6xHis, GST, etc.). In some embodiments, the nucleosome substrates contain an epitope tag, wherein the epitope is the target for antibody production.

In some embodiments, antibody efficiency is determined by performing a genomic mapping assay using a specific low cell number (or a range of cell numbers) to determine antibody signal-to-noise at a range of cell number inputs. The higher efficiency antibodies identified by the present methods generate greater signal at lower cell inputs vs. lower efficiency antibodies, with the higher efficiency antibodies predicted to exhibit greater affinity and/or avidity (see Example 2). It is important to note that antibody/antibody fragment efficiency is challenging to assess when using higher cell numbers, as differences in data quality using a high efficiency antibody or low efficiency antibody are not generally observed. Thus, determining ‘in-application’ antibody/antibody fragment efficiency by utilizing spike-in nucleosomes or performing assays at a range of cell inputs generates data quality that allows assessment of antibody efficiency.

In an embodiment, the candidate antibodies or antibody fragments can be characterized using recombinant designer nucleosome (dNuc) technology at one or more steps of methods for identifying a high efficiency antibody or antibody fragment that targets a histone PTM, which enables characterization of candidate antibody clones binding against physiological nucleosome substrates carrying histone PTMs and/or DNA modifications. In some embodiments, each antibody or antibody fragment targets a different histone PTM and the recombinant nucleosome substrate comprises each of the PTMs. Designer nucleosomes carry defined covalent modifications of the histone proteins (e.g., lysine acylation) or wrapping DNA (e.g., 5′ methylcytosine). In some embodiments the dNucs are designed to contain physiological modifications that are suitable for enzyme assays and high-throughput screenings and comprise a stably positioned nucleosome, and provide a desirable substrate for a range of assays. In some embodiments, the recombinant nucleosomes contain one or more histone modification and/or DNA modification.

In some embodiments, the genomic mapping assay comprises nucleosome spike-in controls that comprise on-target and off-target epitopes. Adding a nucleosome panel of controls can be performed to thereby enable optional quality control testing at one or more, or each, subsequent step of the assay.

The genomic assay can comprise evaluating antibody or antibody fragment binding efficiency at a range of cell inputs. In some embodiments, the identified high efficiency antibody or antibody fragment generates substantially similar data at 500,000 cells and at fewer than 100,000, 50,000 25,000 10,000, 5000, 1000, 100, or 10 cells. Substantially similar data as used herein is defined as within 10%, 9%, 8%, 7%, 6%, 5%, 4%, 3%, 2%, 1% or less difference in the quality of data. In some embodiments, the data quality is determined using Fragment of Reads in Peaks (FRiP) score or replicate analysis (i.e., Pearson correlation).

In some embodiments, binding efficiency of a genomic assay is calculated utilizing spiked-in dNucs and E. coli genomic DNA. In an embodiment, dNucs are spiked in at the beginning of the assay and the E. coli genomic DNA just prior to library preparation, wherein the amount of E. coli DNA sequenced enables normalization between reactions and antibody efficiency can be calculated as on-target dNuc recovered divided by the amount of E. coli DNA.

In some embodiments, the antibody or antibody fragment is a polyclonal antibody or antibody fragment, monoclonal antibody or antibody fragment, recombinant polyclonal antibody or antibody fragment, recombinant monoclonal antibody or antibody fragment, nanobody, antigen-binding fragments (Fab), single domain antibodies (sdAbs), or single chain variable fragments (scFvs).

In an embodiment, optimization of antibodies or antibody fragments, including the high efficiency antibodies described herein, comprise use of recombinant modified nucleosomes, carrying one or more chromatin elements, e.g., histone and/or DNA modifications, histone mutations, histone variants, or proteins that directly or indirectly bind chromatin, to systematically identify antibodies for chromatin assays. In some embodiments, recombinant nucleosome substrates are utilized to identify high efficiency antibodies.

In some embodiments, antibodies or antibody fragments are used in high-throughput dCypher® assays to profile the binding activity of antibody or antibody fragment against a panel of DNA-barcoded designer nucleosomes (dNucs) carrying a diverse array of PTMs. In some embodiments the assay is a binding assay, which is any assay that measures interactions between two molecules, in this instance, an assay that measures the binding of a recombinant fusion protein. In an embodiment, the binding assay may be, for example, a bead-based proximity assay or bead-based multiplex assay bead-based assay, wherein antibodies or antibody fragments are profiled against a panel of dNucs coupled to spectrally barcoded Luminex xMAP beads for rapid evaluation of binding activity to multiple targets in a single reaction.

In some embodiments, a high efficiency antibody or antibody fragment identified by the methods described herein is also provided. In an embodiment, the identified antibody or antibody fragment can be used in a genomics assay, which includes assays for identification, comparison, or measurement of genomic features including DNA sequence, structural variation, gene expression, and gene function, and including chromatin assays. In some embodiments, the high efficiency antibody or high efficiency antibody fragment provides improved signal to noise ratio in low input genomics assays relative to commercially available antibodies. In an embodiment, the high efficiency antibody or high efficiency antibody fragment identified by the methods herein improve assay sensitivity and/or efficiency. Example chromatin assays that use the high efficiency antibody or high efficiency antibody fragment of the present invention include ChIP-seq, Cleavage under targets and release using nuclease (CUT&RUN) (Skene et al., (2017) An efficient targeted nuclease strategy for high-resolution mapping of DNA binding sites eLife 6: e21856; doi: 10.7554/eLife.21856), Cleavage under targets and tagmentation (CUT&Tag) (Kaya-Okur, et al. CUT&Tag for efficient epigenomic profiling of small samples and single cells. Nat Commm 10, 1930 (2019); doi: 10.1038/s41467-019-09982-5) or any other genomics assay known in the art including those as described in International Patent Publication Nos. WO 2019/060907 and WO 2018/042251, incorporated herein by reference in their entirety. Samples for chromatin assays include cells, nuclei, or biological fluids (e.g., cell-free nucleosomes). Biological samples for genomic assays performed on biological fluids, can include, for example, samples of blood, plasma, saliva, stool, and spinal fluid. In an aspect, the genomic mapping assay is performed on<100,000 cells, <90,000 cells, <80,000 cells, <70,000 cells, <60,000 cells, <55,000 cells, <50,000 cells, <40,000 cells, <30,000 cells, <20,000 cells, <10,000 cells, <1,000-2,000 cells, <500 cells, <10⁰ cells, <10 cells, or on a single cell.

Example genomic mapping assays useful with the invention include CUT&RUN, CUT&Tag, and ChIP-seq. For ChIP seq, steps include crosslinking the DNA and the protein in live cells, lysing the cells and extracting and shearing the chromatin, immunoprecipitating with the high efficiency antibody or high efficiency antibody fragment targeting the protein of interest, followed by extracting the DNA from the protein. Evaluating the chromatin elements can be performed at specific regions of interest within the genome by quantitative PCR (qPCR) or genome-wide by Next Generation Sequencing.

In some embodiments, antibodies or antibody fragments identified by the methods described herein exhibit an increase in nucleosome capture efficiency relative to commercial antibodies, for example, current best-in-class antibodies that were selected using histone peptides, for example, 2-fold, 3-fold, 4-fold, 5-fold, 6-fold, 7-fold, 8-fold, 9-fold, or 10-fold higher nucleosome capture efficiency. In embodiments, the antibodies or antibody fragments generate improved signal to noise in low input CUT&Tag assays. In some embodiments, a high efficiency antibody or high efficiency antibody fragment has an increased binding efficiency of 20%, 30%, 40%, 50%, 60%, 70%, 80%, or more increase in binding efficiency to a nucleosome relative to antibodies developed using histone peptides, for example, current commercially available antibodies.

In some embodiments, a DNA-barcoded nucleosome panel can be designed. In an embodiment, the high efficiency antibody or high efficiency antibody fragment can be utilized in genomic mapping assays such as CUT&RUN and CUT&Tag. In an embodiment, the high efficiency antibody or high efficiency antibody fragment improves assay sensitivity and/or efficiency. The methods provided herein can comprise a high efficiency antibody or high efficiency antibody fragment with an increase in binding efficiency relative to antibodies developed using histone peptides, for example, current commercially available antibodies. Nucleosomes useful in the present invention include recombinant nucleosome

substrates and DNA-barcoded nucleosomes as spike-in controls for epigenomic mapping assays. Example DNA-barcoded nucleosomes as spike-in controls for epigenomic assays include those described in International Patent Publication Nos. WO 2020/132388, WO 2020/168151, WO 2019/140082, WO 2013/184930, WO 2015/117145, each of which is incorporated herein in their entirety.

A recombinant nucleosome can comprise one or more chromatin elements. Chromatin elements as used herein include histone modifications, histone mutations or histone variants, DNA modifications, or proteins that directly or indirectly bind chromatin. In some embodiments, the chromatin elements comprise proteins that bind unmodified histones. The chromatin elements can be comprised of both histone modifications and proteins that directly or indirectly bind chromatin.

A recombinant nucleosome substrate may comprise a recombinant mononucleosome. The recombinant nucleosome substrate may comprise one or more histone modification and/or DNA modification, i.e., is functionalized. A recombinant nucleosome may comprise a protein octamer, containing two copies each of histones H2A, H2B, H3, and H4, and optionally, linker histone HI. Each of the histones in the nucleosome is independently fully synthetic, semisynthetic, or recombinant. Methods of producing histones synthetically, semi-synthetically, or recombinantly are well known in the art.

In an aspect, the histone can comprise one or more post-translational modification. The histone PTM may be any PTM for which measurement is desirable. In some embodiments, the histone PTM is, without limitation, N-acetylation of serine and alanine; phosphorylation of serine, threonine and tyrosine; N-crotonylation, N-acylation of lysine; N6-methylation, N6,N6-dimethylation, N6,N6,N6-trimethylation of lysine; omega-N-methylation, symmetrical-dimethylation, asymmetrical-dimethylation of arginine; citrullination of arginine; ubiquitinylation of lysine; sumoylation of lysine; O-methylation of serine and threonine, ADP-ribosylation of arginine, aspartic acid and glutamic acid, or any combination thereof.

In one embodiment, the post translational modification is selected from one or a combination of modifications listed in Tables 1 (a)-1 (f) of International Patent Publication WO 2019/169263, specifically incorporated herein by reference.

The histone mutation may be any mutation known in the art or any mutation of interest. In some embodiments, the histone mutations are oncogenic mutations, e.g., mutations associated with one or more types of cancer. Known oncogenic histone mutations include, without limitation, H3K4M, H3K9M, H3K27M, H3G34R, H3G34V, H3G34W, H3K36M, or any combination thereof.

Several naturally occurring histone variants are known in the art and any one or more of them may be included in the nucleosome. Histone variants include, without limitation, H3.3, H2A.Bbd, H2A.Z.1, H2A.Z.2, H2A.X, mH2A1.1, mH2A1.2, mH2A2, TH2B, or any combination thereof.

Example chromatin binding proteins include transcription factors (protein family members of helix-turn-helix, helix-loop-helix, zinc finger, basic protein-leucine zipper, and beta-sheet motifs, for example, CTCF 2, 4, 5, FoxA1/FoxA2 5, and OCT4 4, 5), PTM binding effectors (e.g., kinase inhibitors, ubiquitin protease, E3 ubiquitin ligase; acetyltransferase, phosphotheonine lyase; protein kinase) and chromatin remodelers (e.g., proteins of remodeler families SWI/SNF (switch/sucrose-non-fermenting), ISWI (imitation switch), CHD (chromodomain-helicase-DNA binding) and INO80 (inositol requiring 80)).

The DNA post-transcriptional modification may be any modification for which measurement is desirable. Known post-transcriptional DNA modifications include, without limitation, 5-methylcytosine, 5-hydroxymethylcytosine, 5-formylcytosine, 5-carboxylcytosine, 3-methylcytosine, 5,6-dihydrouracil, 7-methylguanosine, xanthosine, and inosine. In some embodiments, the DNA post-transcriptional modification is 5-methylcytosine, 5-hydroxymethylcytosine, 5-formylcytosine, 5-carboxylcytosine, 3-methylcytosine, or any combination thereof.

The recombinant mononucleosome may comprise a mix of recombinant and/or synthetic histone octamers, one or more of which may comprise post-translational modifications (PTMs). In some embodiments, the recombinant nucleosomes are polynucleosomes comprising a plurality of octamers. In an aspect, the polynucleosome comprises less than 6, 5, 4, or 3 histone octamers. In some embodiments, each histone octamer comprises the same PTM(s), e.g., the nucleosomes are homogenous. In other embodiments each histone octamer comprises different PTM(s), e.g., the nucleosomes are heterogeneous. The recombinant mononucleosomes can be as described in International Patent Publication No. WO 2018/213719, incorporated herein by reference in its entirety. In an embodiment, recombinant substrates can be manufactured to contain one (or more) physiological or disease-relevant histone and/or DNA modifications.

Pools or panels of DNA-barcoded designer nucleosomes (dNucs) carrying on- and related off-target epitopes (e.g., histone PTMs or chromatin binding domain epitopes) may be used as controls to directly assess antibody or antibody fragment performance. The barcode sequence and methods of incorporation and use can be as described in International Patent Publication No. WO 2019/140082 and International Patent Publication WO 2020/132388, incorporated herein by reference. Barcoding can be used as needed to identify the cleaved nucleosomal DNA, for example by sample, individual, or other source identifying information.

In some embodiments, the method for screening a detection reagent (e.g., a high efficiency antibody or high efficiency antibody fragment) in an optimization assay comprises use of a panel (e.g., a plurality) of spike-in nucleosomes. The number of different nucleosomes in a panel can include a plurality of species, which may include duplicates of each standard having distinct barcode identifier sequences as a form of internal control. Thus, the panel may include species represented multiple times at the same or different concentrations with each standard having a unique barcode identifier sequence that represents the concentration of the standard. For example, each standard may be present in the panel in 2, 3, 4, 5, 6, 7, 8, 9, 10 or more different concentrations, each concentration having a different barcode identifier sequence. Thus, a panel may have unique standards of between about 5 and about 250 total species. Example methods of use are described in the working examples, as well as International Patent Publication WO 2019/140082, incorporated herein by reference in its entirety.

The present invention also relates to leveraging these developed high efficiency antibodies and antibody fragments to expand the application of low input, SC, or spatial chromatin assays that use antibodies to detect chromatin and/or nucleosome substrates in vivo or in vitro. These high efficiency antibodies will provide first-time access to chromatin targets (e.g., histone PTMs, transcription factors, and other chromatin binding proteins) in assays that use limited cells or even SCs. For example, the use of high efficiency antibodies and antibody fragments with ultra-high binding affinity in a nucleosome context improves the sensitivity of chromatin profiling assays, including ChIP-seq, CUT&RUN, and/or CUT&Tag assays, allowing investigators to use fewer cells per reaction (including SC) by generating more data per cell. For example, Applicant has found that >60% of current best-in-class PTM antibodies (n=12) fail to generate sufficient data quality using <10K primary cells; Table 1 shows representative data. Of note, DNA yields from 10K B cells were substantially lower vs. 10K K562 cells (30-40x), highlighting the sensitivity challenges associated with genomic mapping from primary immune cells. Other assays that could benefit from these high efficiency binding affinity antibodies (via increased assay sensitivity) include those that measure circulating nucleosomes from biological fluids (e.g., blood, plasma, serum, urine, cerebral spinal fluid, feces, and lymph).

TABLE 1
CUT&RUN using best-in-class antibodies across cell types/inputs.

| Target | 500K K562 | 10K K562 | 10K B Cell |
|---|---|---|---|
| H3K4me3 | Pass | Pass | Pass |
| H3K4me1 | Pass | Pass | Pass |
| H3K27me3 | Pass | Pass | Pass |
| H3K36me3 | Pass | Pass | Pass |
| H3K36me3 | Pass* | Pass | Fail |
| H3K36me3 | Pass* | Pass | Fail |
| H3K36me3 | Pass | Fail | Fail |
| H3K36me3 | Pass | Fail | Fail |
| H3K36me3 | Pass | Fail | Fail** |
| H3K9me3 | Pass | Pass | Fail |
| H3K9me3 | Pass | Fail | Fail |

“Pass”: FRiP >0.05, <20% off-target dNuc recovery.
*= antibody not tested in 500K cells, assumed to pass given data at 10K.
**= antibody not tested in 10K B cells.

A further aspect of the invention relates to kits for use in the methods of the invention. Kits may comprise one or more high efficiency antibodies or high efficiency antibody fragments; optionally nucleosome spike-ins, one or more solid supports; and/or instructions for use, in any combination. The kit can further comprise modulating agents, carriers, buffers, containers, devices for administration of the components, and the like. The kit can further comprise labels and/or instructions for assay selection and execution. Such labeling and/or instructions can include, for example, information concerning the amount, and method of administration, detection and quantification for the assays detailed herein.

Having described the present invention, the same will be explained in greater detail in the following examples, which are included herein for illustration purposes only, and which are not intended to be limiting to the invention.

### Examples

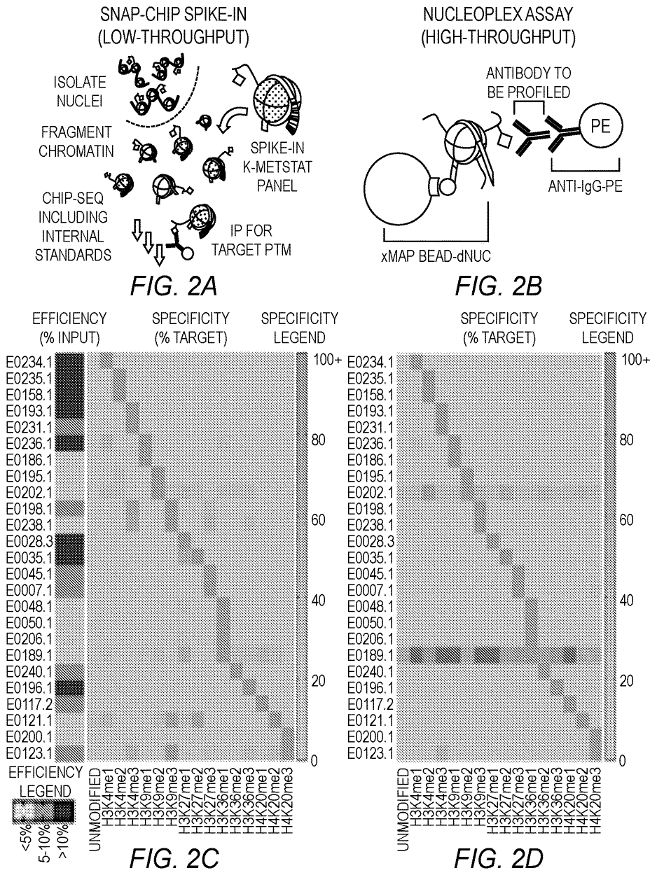

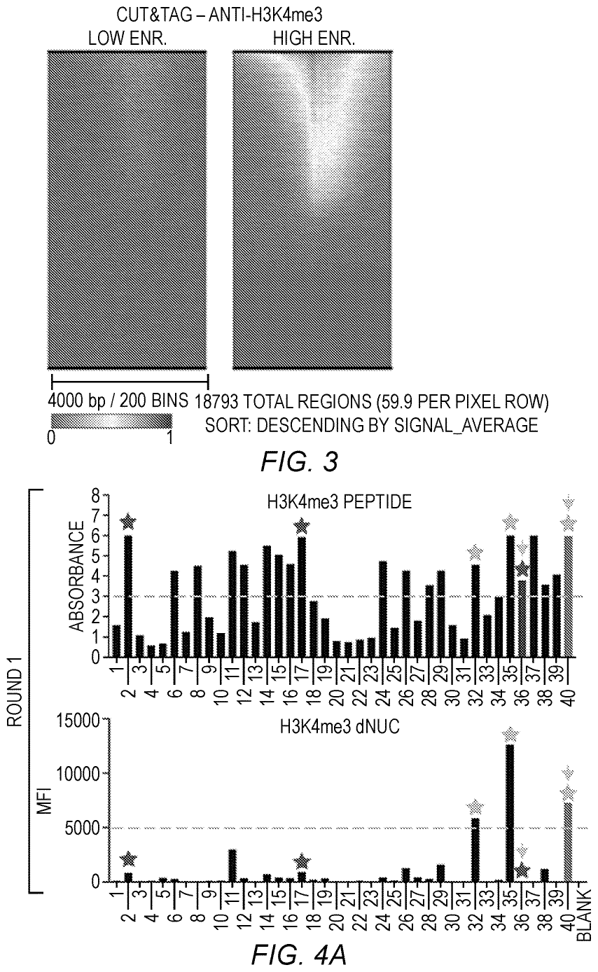

Antibodies are a key reagent for low input/SC genomic mapping studies, but many antibodies lack the requisite specificity and efficiency needed for these sensitive applications. It is well established that the accuracy of antibodies is an increasing concern in the biomedical field (Egelhofer, Minoda et al. 2011, Fuchs and Strahl 2011, Nishikori, Hattori et al. 2012, Baker 2015, Bradbury and Plückthun 2015, Shah, Grzybowski et al. 2018), as >$750 million is spent worldwide each year on nonspecific or “bad” antibodies (Bradbury and Plückthun 2015). This is particularly true for histone PTM antibodies, which are plagued by poor specificity and low enrichment for chromatin mapping studies (Fuchs, Krajewski et al. 2011, Fuchs and Strahl 2011, Rothbart, Lin et al. 2012, Shah, Grzybowski et al. 2018). To begin to address this, EpiCypher (Durham, NC) has commercialized DNA-barcoded nucleosome spike-in panels for genomic map studies (including ChIP-seq, CUT&RUN, and CUT&Tag; products termed SNAP-ChIP, SNAP-CUTANA, SNAP-CUT&RUN, and SNAP-CUT&Tag), comprised of pools of on- and related off-target PTM-carrying DNA-barcoded designer nucleosomes (dNucs) that enable in-assay monitoring of antibody performance (FIG. 2A). This platform technology was based on prior work by EpiCypher and others, developing DNA-barcoded nucleosomes for multiplexed binding assays (International Patent Publication No. WO 2013184930) and genomic mapping assays (International Patent Publication Nos. WO 2015117145; WO 2019140082; WO 2020132388; WO 2020132388), each of which are incorporated herein by reference.

While recombinant nucleosome spike-ins are a powerful technology to determine in-assay antibody specificity and efficiency, this approach is low-throughput and costly, thereby limiting its utility for antibody development. Thus, to bridge this gap, EpiCypher also developed a high-throughput multiplex assay that uses nucleosome-based substrates (dCypher®-Luminex), delivering the first high-throughput nucleosome-based solution for antibody specificity profiling. This approach couples dNucs to spectrally barcoded Luminex xMAP® beads for multiplexed (up to 500 targets) high-throughput antibody screening (FIG. 2B). Importantly, dCypher-Luminex generates specificity data highly predictive of SNAP spike-ins at a fraction of the time and cost (FIGS. 2C vs. 2D). However, dCypher-Luminex is only able to predict antibody specificity, not efficiency. Thus, using these technologies together, antibody clone specificity can be rapidly screened using dCypher-Luminex, and those that exhibit high specificity can then be validated in-assay using SNAP spike-ins. This dual-screening strategy was applied to test >400 commercial histone PTM antibodies and found that >70% displayed unacceptable off-target binding and/or low efficiency (FIGS. 2C, 2D). Antibody binding on histone peptides (current gold-standard) was also found to have minimal correlation with binding specificity in a nucleosome context (52 antibodies; R2=0.2337) (Shah, Grzybowski et al. 2018), together demonstrating nucleosome-based technologies are needed for histone PTM antibody validation.

Inspired by the massive commercial antibody screen, a histone PTM antibody development pipeline was developed that leverages recombinant nucleosome technology during each step of antibody selection (FIG. 1), with a goal of developing ultra-efficient antibodies for chromatin mapping studies Antibody selection using nucleosome substrates was determined as key to identification of clones that exhibit high affinity for histone PTMs in the physiologically relevant chromatin context, as provided in the representative examples of novel antibody development pipeline used to generate ultra-efficient recombinant antibodies to histone PTMs. First, peripheral blood mononuclear cells (PBMCs) from immunized animals were screened by ELISA for PTM recognition using dNucs (Round 1). Second, selected PBMC clones from Round 1 were isolated, subcloned, and expressed in cell lines, and resulting supernatants were then screened using Luminex-dCypher assay (Round 2). Third, top candidates from Round 2 were purified and analyzed for antibody specificity/efficiency using DNA-barcoded SNAP spike-ins directly in a preferred genomic mapping assay (e.g., ChIP-seq, CUT&RUN, CUT&Tag, etc., Round 3). Optionally, antibody efficiency can be determined by testing the antibody at a range of cell inputs and determining if the antibody is capable of generating sufficient data quality at low cell numbers. Antibodies were at a range of cell inputs from 500k to 5K cells to determine antibody efficiency. Antibodies that generated similar data using at 500K and 50K cells were determined to be high efficiency.

As demonstrated in the following examples, the novel antibody development pipeline generates clones that exhibit a >5-10× increase in nucleosome capture efficiency (vs. current best-in-class antibodies that were selected using histone peptides) (see Example 1). Significantly, the ultra-efficient antibodies generated improved S/N in low input CUT&Tag assays (see Example 2).

### Example 1. Physiological Screening Substrates are Key to Identifying Highly-Specific High Efficiency Antibodies

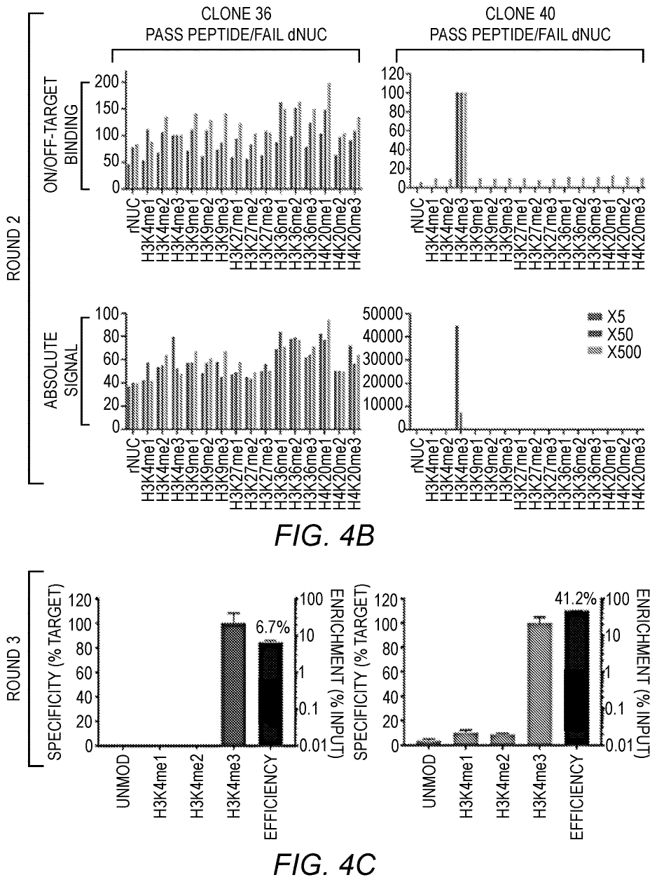

Most antibody development pipelines use modified histone peptides to assess on-target recognition and specificity. However, as has been shown, antibody binding to histone peptides does not correlate with specificity in genomic mapping assays (Shah, Grzybowski et al. 2018). This inspired the development of the dNuc-based antibody screening platform (FIGS. 2A-2D), which was employed here to identify antibodies to two high-value histone PTM targets: H3K4me1 and H3K4me3 (FIGS. 4A-4C). Screening candidate clones using dNucs according to this method identified antibodies with 5-10× greater binding efficiency than current best-in-class commercial antibodies (FIGS. 4A-4C).

For these studies, rabbits were immunized with H3K4me1 or H3K4me3 peptides. Round 1 screening of PBMCs from these rabbits using either histone peptides or dNucs (e.g. H3K4me3; FIG. 4A) was then performed. It was found that screening against histone peptides poorly correlated with binding to dNucs (FIG. 4A). Indeed, some candidate clones that passed peptide screening (see grey dashed line) generated very low signal on dNucs (FIG. 4A, black stars). However, some clones were identified that generated very high S/N when targeting both histone and dNuc substrates (FIG. 4A, gray stars). Thus, the present invention provides a much-improved screening approach (vs peptide-based screening) to identify antibodies that have high binding affinity for an epitope in a nucleosome context, which has the potential to dramatically reduce the screening costs in order to identify top performing clones.

To directly test if performance in peptide and dNuc screening assays was predictive of performance in the dCypher-Luminex assay, we subcloned and performed Round 2 dCypher-Luminex screening on clones that either passed peptide screening and failed dNuc screening (e.g., Clone 36) or clones that passed both peptide and dNuc screening (e.g., Clone 40) (FIG. 4A, arrows). In the dCypher-Luminex assay, biotinylated dNucs carrying on- and off-target PTMs are coupled to spectrally barcoded, streptavidin-coated Luminex xMAP beads (FIG. 2B). Each antibody clone was mixed with the dNuc panel (K-MetStat panel; comprised of 16 dNucs carrying various lysine methylation moieties) and binding was quantified by adding an anti-rabbit secondary antibody conjugated to phycoerythrin, which is detected using the En Vision detection system (Luminex). For these studies, each antibody clone was probed using three different antibody concentrations (1:5; 1:50; 1:100). It was found that the candidate clone that failed dNuc screening by ELISA also exhibited high off-target activity and low dNuc recovery in dCypher-Luminex, demonstrating its inability to bind the PTM in a nucleosomal context (FIG. 4B). By contrast, the clone that exhibited high dNuc binding by ELISA displayed high target binding and exquisite target specificity (FIG. 4B). Finally, these two clones were purified for Round 3 profiling and validation in SNAP-ChIP. As expected, the clone that exhibited high dNuc binding in ELISA/NucleoPlex testing also showed exceptionally high efficiency by SNAP-ChIP (41.2%; FIG. 4C). Conversely, the clone that performed poorly in Round 1/2 screening (and would have failed the screening standards) exhibited relatively low efficiency (6.7%; FIG. 4C).

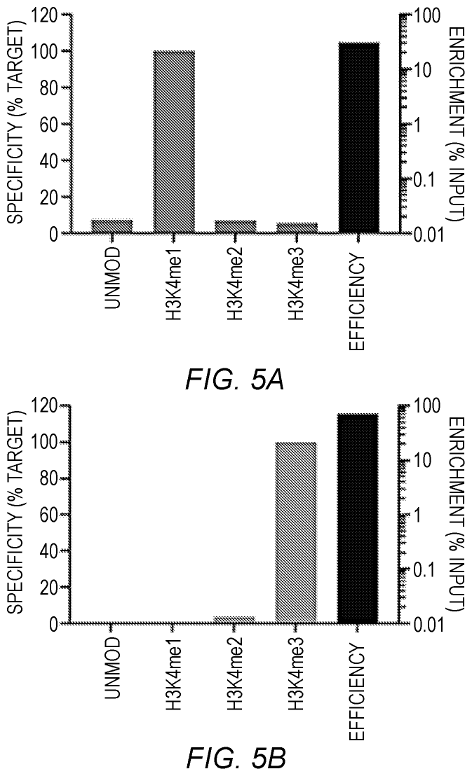

Using the antibody development scheme above, hundreds of clones for H3K4me1 and H3K4me3 were successfully screened. Ultimately, antibodies to both H3K4me1 and H3K4me3 were identified that exhibited exquisite target specificity and ultra-high binding efficiency (FIGS. 5A, 5B). Remarkably, this approach identified candidate clones with binding efficiencies for H3K4me1 (31%) and H3K4me3 (71%) that far exceed typical efficiency profiles (by 5-10x) of current best-in-class antibodies. Indeed, based on prior work using SNAP-ChIP, efficiency scores for histone PTM antibodies generally range between 1-10% (Shah, Grzybowski et al. 2018).

### Example 2: Development of Improved Genomic Mapping Assays Using Ultra-Efficient Antibodies

Here, the application of identified high efficiency (HE) antibodies to H3K4me1 and H3K4me3 from Example 1 were tested head-to-head with existing commercial antibodies in CUT&RUN and found that HE antibodies are required to enable ultra-low input genomic mapping for both H3K4me1 and H3K4me3. CUT&RUN was performed in unfixed K562 cells using the antibodies to high and low efficiency antibodies to H3K4me1 and H3K4me3. Unfixed K562 cells were immbolized on Concanavalin A (ConA) beads and permeabilized. Antibodies to H3K4me1 or H3K4me3 (0.5 μg) were added, followed by a protein A/G-micrococcal nuclease (pAG-MNase) fusion protein. pAG-MNase was activated by addition of calcium to cleave and release antibody-bound chromatin; then supernatant was collected and libraries were prepared and sequenced to 10M reads. Data analysis was performed using an internal analysis pipeline, which is based on published work (Skene, Henikoff et al. 2018, Meers, Bryson et al. 2019, Meers, Tenenbaum et al. 2019). Briefly, raw reads were mapped to the reference genome using Bowtie2 (Langmead and Salzberg 2012). Resulting SAM files were filtered using SAMtools (Li, Handsaker et al. 2009), and BEDTools (Quinlan and Hall 2010) was used to create genome coverage BEDgraphs. To parse the data, SEACR was used to call peaks (Meers, Tenenbaum et al. 2019).

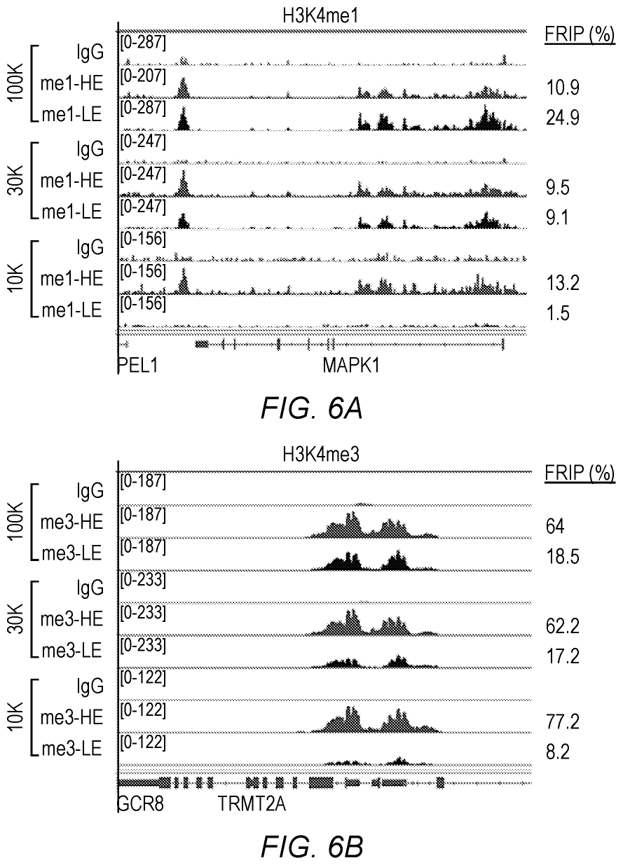

HE antibodies were found essential to generate sufficient S/N in low input (<30K cells) CUT&RUN experiments. At 100K cells, IGV tracks resulting from the HE and competitor low-efficiency (LE) antibodies yielded comparable peak structures and S/N (Fragment of Reads in Peaks; FRiP %) for both H3K4me1 and H3K4me3 (FIGS. 6A, 6B). HE antibodies maintained high S/N at all cell inputs tested, whereas signal from LE antibodies was dramatically reduced as fewer cells were used in the assay, with essentially no signal detected at 10K cells (FIGS. 6A, 6B). Indeed, HE antibodies yielded nearly 10× higher S/N (i.e., FRiP %) at 10K cells than LE antibodies for both H3K4me1 and H3K4me3 (FIGS. 6A, 6B), demonstrating the importance of considering antibody efficiency when performing low input genomic mapping studies.

### Example 3: Development of Methods to Determine Antibody Efficiency in Immunotethering-Based Genomic Assays (e.g., CUT&RUN/CUT&Tag)

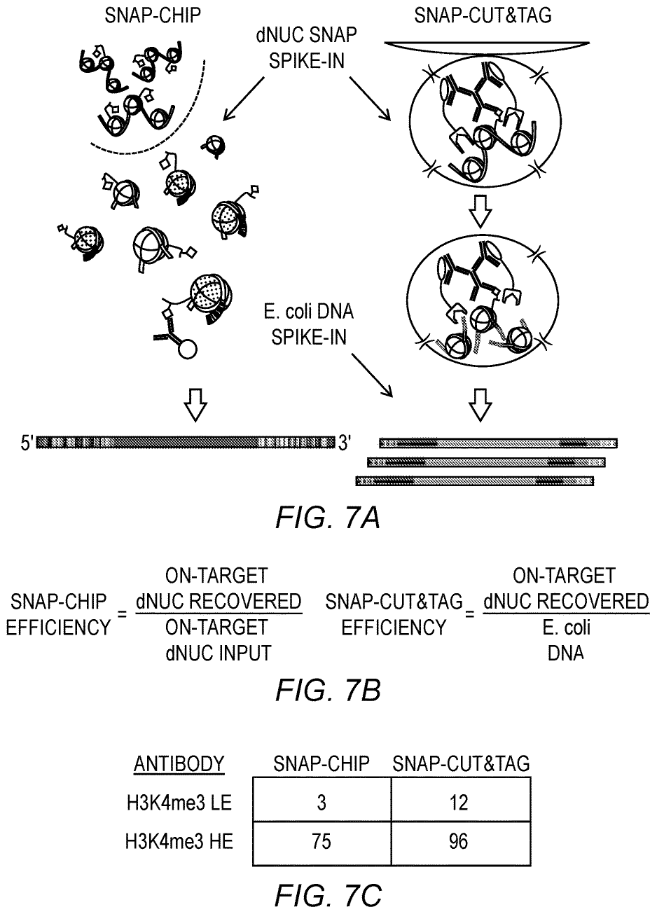

The dual dCypher-Luminex (Round 2)/SNAP spike-in (Round 3) screening approach is a key pillar of this innovative antibody development pipeline. Here, the antibody development pipeline was improved by developing a novel method to calculate antibody efficiency using SNAP spike-ins in a CUT&Tag workflow (an assay also referred to SNAP-CUT&Tag™). In SNAP-ChIP, antibody efficiency is calculated by taking the ratio of the number of immunoprecipitated spike-in dNucs (read out by qPCR) to the number of total spike-in dNucs added to the reaction (FIGS. 7A-7C). However, because in CUT&Tag antibody binding and tagmentation happen in situ, there is no reference input in this assay. To calculate binding efficiency in SNAP-CUT&Tag, dNucs were spiked-in at the beginning of the assay as well as a E. coli genomic DNA just prior to library prep (FIGS. 7A-7C). The libraries are then sequenced, and the amount of E. coli DNA sequenced enables normalization between reactions (FIGS. 7A-7C), acting similar to input DNA in the SNAP-ChIP workflow. Thus, in SNAP-CUT&Tag, antibody efficiency equals on-target dNuc recovered divided by the amount of E. coli DNA (FIGS. 7A-7C). Antibody efficiencies calculated via SNAP-CUT&Tag were found comparable to those calculated via SNAP-ChIP (FIGS. 7A-7C). This new method permits calculation of antibody efficiency in CUT&Tag- and CUT&RUN-based workflows.

### Example 4: Development of H3K36Me2 Antibody

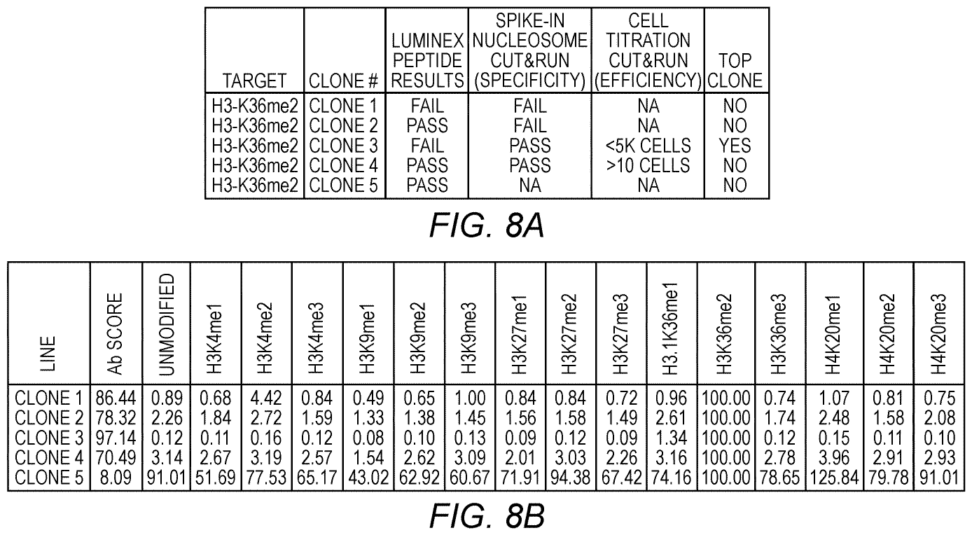

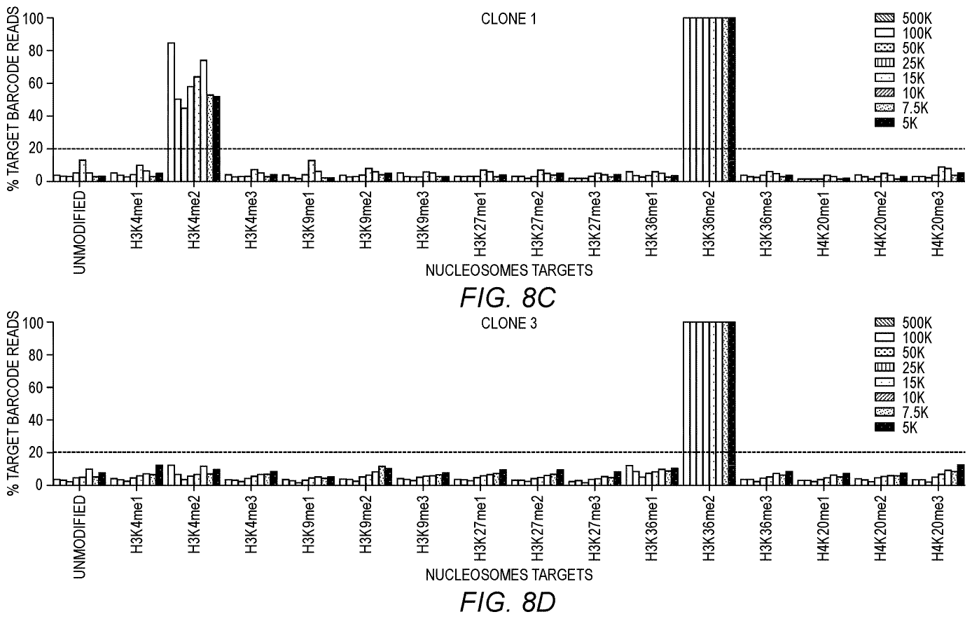

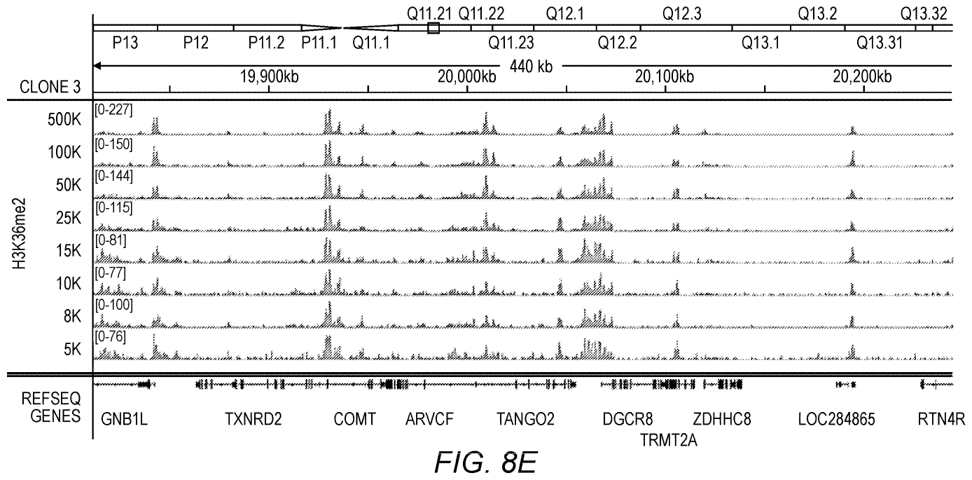

A central tenet of the present invention is the use of recombinant nucleosome substrates during antibody clone selection. In this novel antibody development workflow, recombinant nucleosome substrates are used in place of peptide-based substrates—the current approach widely used in the art. Here, we directly asked if profiling of candidate clones using nucleosomes can identify unique clones that would be missed using peptides. Unique clones identified using nucleosome-based profiling were examined for their ability to outperform current best-in-class reagents. Screening candidate clones using nucleosomes identified unique clones that were not identified by histone peptide. It was also found that an antibody clone that was only identified by nucleosomes (and not peptide) exhibited high efficiency in CUT&RUN assays, generating high quality data using as few as 5K cells (FIGS. 8A-8E). Finally, the performance of this high efficiency H3K36me2 antibody was compared with the current best-in-class H3K36me2 commercial antibody. These data show that the high efficiency antibody can generate improved assay signal at 50K cells compared to the current best in class antibody (FIG. 8, 9). Together, these data demonstrate how the present invention can be used to generate improved high efficiency chromatin targeting antibodies, highlighting the novel result that substrate choice (i.e., nucleosome vs peptide) has a profound impact on the identification of antibody clones with improve assay performance. We surmise screening candidate clones using nucleosome substrates allows for the identification of clones that can efficiently bind the target motif in a nucleosome context. By contrast, peptide-based screening approaches can identify clones that are specific for the target, but this surrogate assay is insufficient to identify clones that exhibit high binding efficiency in a nucleosomal context.

In this study the goal was to develop a recombinant monoclonal antibody to H3K36me2. 4 rabbits were first immunized with an H3K36me2-containing modified histone peptide. Next, B cells from the rabbit with the most robust antigenic response were isolated and the B cell sera profiled using a panel of modified histone peptides or recombinant nucleosomes; the panels included mono-, di- and tri-methylation for H3K4, H3K9, H3K27, H3K36, H4K20 as well as unmodified substrates as negative controls. These assays were performed using a Luminex-based assay, wherein each histone peptide or nucleosome in the panel was conjugated to a unique spectrally barcoded magnetic xMap bead. Representative data from this screen in shown in FIG. 8A. These data demonstrate that substrate choice results in the identification of different candidate clones to move forward for further testing. Based on this initial screen, 12 candidates were cloned, expressed, and purified based on testing results with their respective sera. In this group a collection of clones was selected that passed initial testing using histone peptides and/or nucleosomes. These antibody clones were first validated by rescreening each clone using the nucleosome-based Luminex-based described above (dCypher-Luminex). Representative data from this screen is shown in FIG. 8B, which essentially recapitulates our initial screen and demonstrates successful cloning and expression of each candidate clone. The clone that was selected based on its strong binding to H3K36me2 peptides showed poor target specificity for nucleosomes (see FIG. 8B; clone 5). Based on these results, a 1 mg lot of each of the five best performing antibodies were generated for final ‘in application’ validation in CUT&RUN. For this final test, each antibody was testing in CUT&RUN using a range of cell inputs (500K-5K cells) to assess antibody efficiency. Importantly, each CUT&RUN reaction also included DNA-barcoded spike-in nucleosomes to quantitatively determine antibody specificity at each experimental condition (i.e., cell input number). Of the five antibodies selected, two showed high target specificity based on nucleosome spike-in controls. FIGS. 8C-8D show representative example of two antibodies with low (FIG. 8C) or high (FIG. 8D) target specificity as determined by spike-in nucleosome controls. The two antibodies that passed specificity testing were both highly efficient, generating high quality data using as few as 5K or 10K cells. FIG. 8E shows cell titration from the top performing antibody identified from this study (Clone 3), showing that this antibody can generate high quality data using <5K cells.

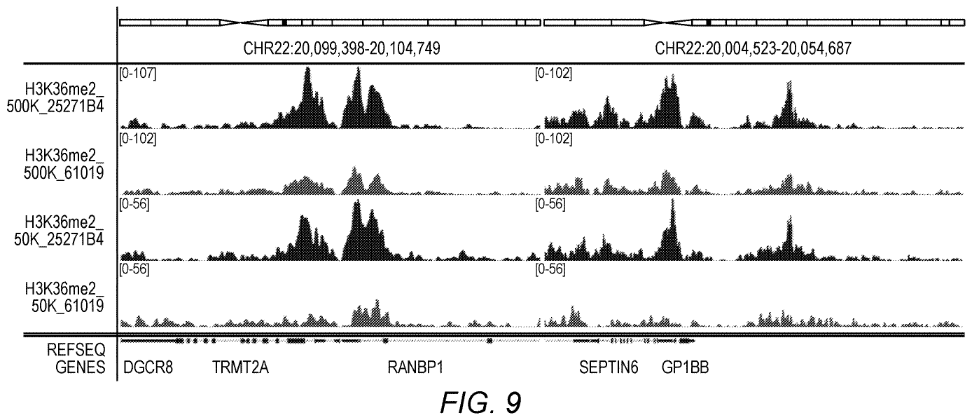

Finally, the high efficiency H3K36me2 antibody (Clone 3 from FIGS. 8A-8E) was benchmarked against the best-in-class commercial H3K36me2 antibody (Active Motif; Cat #61019) in low input CUT&RUN assays. For this experiment, CUT&RUN reactions were performed using 500K cells (high input) or 50K (low input) cells to directly compare antibody efficiency (FIG. 9). As expected, the high efficiency H3K36me2 antibody generates similar, high-quality data at both 500K (H3K36me2_500K_25271B4; aka Clone 3) or 50K (H3K36me2_50K_25271B4; aka Clone 3) cells; however, the commercial H3K36me2 antibody exhibits lower assay signal at 500K cells (H3K36me2_500K_61019) and generates very poor data quality at 50K cells (H3K36me2_50K_61019). These data highlight how high efficiency antibodies can be leveraged to improve low input and potentially even single cell genomic mapping applications.

### REFERENCES

Ahn J, Heo S, Lee J, Bang D. Introduction to Single-Cell DNA Methylation Profiling Methods. Biomolecules. 2021 Jul. 10; 11 (7): 1013. doi: 10.3390/biom11071013
Alfaleh, M. A., H. O. Alsaab, A. B. Mahmoud, A. A. Alkayyal, M. L. Jones, S. M. Mahler and A. M. Hashem (2020). “Phage Display Derived Monoclonal Antibodies: From Bench to Bedside.” Front Immunol 11:1986.
Baker, M. (2015). “Reproducibility crisis: Blame it on the antibodies.” Nature 521 (7552): 274-276.
Bradbury, A. and A. Plückthun (2015). “Reproducibility: Standardize antibodies used in research.” Nature 518 (7537): 27-29.
Buenrostro, J. D., P. G. Giresi, L. C. Zaba, H. Y. Chang and W. J. Greenleaf (2013). “Transposition of native chromatin for fast and sensitive epigenomic profiling of open chromatin, DNA-binding proteins and nucleosome position.” Nat Methods 10 (12): 1213-1218.
Deng, Y., M. Bartosovic, P. Kukanja, D. Zhang, Y. Liu, G. Su, A. Enninful, Z. Bai, G. Castelo-Branco and R. Fan (2022). “Spatial-CUT&Tag: Spatially resolved chromatin modification profiling at the cellular level.” Science 375 (6581): 681-686.
Deng, Y., Bartosovic, M., Ma, S. et al. Spatial profiling of chromatin accessibility in mouse and human tissues. Nature 609, 375-383 (2022).
Egelhofer, T. A., A. Minoda, S. Klugman, K. Lee, P. Kolasinska-Zwierz, A. A. Alekseyenko, M. S. Cheung, D. S. Day, S. Gadel, A. A. Gorchakov, T. Gu, P. V. Kharchenko, S. Kuan, I. Latorre, D. Linder-Basso, Y. Luu, Q. Ngo, M. Perry, A. Rechtsteiner, N. C. Riddle, Y. B. Schwartz, G. A. Shanower, A. Vielle, J. Ahringer, S. C. Elgin, M. I. Kuroda, V. Pirrotta, B. Ren, S. Strome, P. J. Park, G. H. Karpen, R. D. Hawkins and J. D. Lieb (2011). “An assessment of histone-modification antibody quality.” Nat Struct Mol Biol 18 (1): 91-93.
Fang, R., Preissl, S., Li, Y. et al. Comprehensive analysis of single cell ATAC-seq data with SnapATAC. Nat Commun 12, 1337 (2021)
Ferreyra Vega, S., Wenger, A., Kling, T. et al. Spatial heterogeneity in DNA methylation and chromosomal alterations in diffuse gliomas and meningiomas. Mod Pathol 35, 1551-1561 (2022).
Fuchs, S. M., K. Krajewski, R. W. Baker, V. L. Miller and B. D. Strahl (2011). “Influence of combinatorial histone modifications on antibody and effector protein recognition.” Curr Biol 21 (1): 53-58.
Fuchs, S. M. and B. D. Strahl (2011). “Antibody recognition of histone post-translational modifications: emerging issues and future prospects.” Epigenomics 3 (3): 247-249. Grosselin, K., A. Durand, J. Marsolier, A. Poitou, E. Marangoni, F. Nemati, A. Dahmani, S. Lameiras, F. Reyal, O. Frenoy, Y. Pousse, M. Reichen, A. Woolfe, C. Brenan, A. D. Griffiths, C. Vallot and A. Gerard (2019). “High-throughput single-cell ChIP-seq identifies heterogeneity of chromatin states in breast cancer.” Nat Genet 51 (6): 1060-1066.
Hainer, S. J., A. Boskovic, K. N. McCannell, O. J. Rando and T. G. Fazzio (2019). “Profiling of Pluripotency Factors in Single Cells and Early Embryos.” Cell 177 (5): 1319-1329 e1311. Harmsen, M. M. and H. J. De Haard (2007). “Properties, production, and applications of camelid single-domain antibody fragments.” Appl Microbiol Biotechnol 77 (1): 13-22.
Kaya-Okur, H. S., S. J. Wu, C. A. Codomo, E. S. Pledger, T. D. Bryson, J. G. Henikoff, K. Ahmad and S. Henikoff (2019). “CUT&Tag for efficient epigenomic profiling of small samples and single cells.” Nat Commun 10 (1): 1930.
Kim, C., R. Gao, E. Sei, R. Brandt, J. Hartman, T. Hatschek, N. Crosetto, T. Foukakis and N. E. Navin (2018). “Chemoresistance Evolution in Triple-Negative Breast Cancer Delineated by Single-Cell Sequencing.” Cell 173 (4): 879-893 e813.
Ku, W. L., K. Nakamura, W. Gao, K. Cui, G. Hu, Q. Tang, B. Ni and K. Zhao (2019). “Single-cell chromatin immunocleavage sequencing (scChIC-seq) to profile histone modification.” Nat Methods 16 (4): 323-325.
Kunert, R. and D. Reinhart (2016). “Advances in recombinant antibody manufacturing.” Appl Microbiol Biotechnol 100 (8): 3451-3461.
Langmead, B. and S. L. Salzberg (2012). “Fast gapped-read alignment with Bowtie 2.” Nat Methods 9 (4): 357-359.
Leenaars, M. and C. F. Hendriksen (2005). “Critical steps in the production of polyclonal and monoclonal antibodies: evaluation and recommendations.” ILAR J 46 (3): 269-279. Li, H., B. Handsaker, A. Wysoker, T. Fennell, J. Ruan, N. Homer, G. Marth, G. Abecasis, R. Durbin and S. Genome Project Data Processing (2009). “The Sequence Alignment/Map format and SAMtools.” Bioinformatics 25 (16): 2078-2079.
Meers, M. P., T. D. Bryson, J. G. Henikoff and S. Henikoff (2019). “Improved CUT&RUN chromatin profiling tools.” Elife 8.
Meers, M. P., D. Tenenbaum and S. Henikoff (2019). “Peak calling by Sparse Enrichment Analysis for CUT&RUN chromatin profiling.” Epigenetics Chromatin 12 (1): 42.
Moffitt, J. R., Lundberg, E. & Heyn, H. The emerging landscape of spatial profiling technologies. Nat Rev Genet 23, 741-759 (2022). https://doi.org/10.1038/s41576-022-00515-3 Nishikori, S., T. Hattori, S. M. Fuchs, N. Yasui, J. Wojcik, A. Koide, B. D. Strahl and S. Koide (2012). “Broad ranges of affinity and specificity of anti-histone antibodies revealed by a quantitative peptide immunoprecipitation assay.” J Mol Biol 424 (5): 391-399.
Peach, S. E., E. L. Rudomin, N. D. Udeshi, S. A. Carr and J. D. Jaffe (2012). “Quantitative assessment of chromatin immunoprecipitation grade antibodies directed against histone modifications reveals patterns of co-occurring marks on histone protein molecules.” Mol Cell Proteomics 11 (5): 128-137.
Plasschaert, L. W., R. Zilionis, R. Choo-Wing, V. Savova, J. Knehr, G. Roma, A. M. Klein and A. B. Jaffe (2018). “A single-cell atlas of the airway epithelium reveals the CFTR-rich pulmonary ionocyte.” Nature 560 (7718): 377-381.
Quinlan, A. R. and I. M. Hall (2010). “BEDTools: a flexible suite of utilities for comparing genomic features.” Bioinformatics 26 (6): 841-842.
Rotem, A., O. Ram, N. Shoresh, R. A. Sperling, A. Goren, D. A. Weitz and B. E. Bernstein (2015). “Single-cell ChIP-seq reveals cell subpopulations defined by chromatin state.” Nat Biotechnol 33 (11): 1165-1172.
Rothbart, S. B., S. Lin, L. M. Britton, K. Krajewski, M. C. Keogh, B. A. Garcia and B. D. Strahl (2012). “Poly-acetylated chromatin signatures are preferred epitopes for site-specific histone H4 acetyl antibodies.” Sci Rep 2:489.
Schmid, M., T. Durussel and U. K. Laemmli (2004). “ChIC and ChEC; genomic mapping of chromatin proteins.” Mol Cell 16 (1): 147-157.
Shah, R. N., A. T. Grzybowski, E. M. Cornett, A. L. Johnstone, B. M. Dickson, B. A. Boone, M. A. Cheek, M. W. Cowles, D. Maryanski, M. J. Meiners, R. L. Tiedemann, R. M. Vaughan, N. Arora, Z. W. Sun, S. B. Rothbart, M. C. Keogh and A. J. Ruthenburg (2018). “Examining the Roles of H3K4 Methylation States with Systematically Characterized Antibodies.” Mol Cell 72 (1): 162-177 e167.
Shah, R. N., A. T. Grzybowski, E. M. Cornett, A. L. Johnstone, B. M. Dickson, B. A. Boone, M. A. Cheek, M. W. Cowles, D. Maryanski, M. J. Meiners, R. L. Tiedemann, R. M. Vaughan, N. Arora, Z. W. Sun, S. B. Rothbart, M. C. Keogh and A. J. Ruthenburg (2018). “Examining the Roles of H3K4 Methylation States with Systematically Characterized Antibodies.” Mol Cell 72 (1): 162-177.e167.
Skene, P. J., J. G. Henikoff and S. Henikoff (2018). “Targeted in situ genome-wide profiling with high efficiency for low cell numbers.” Nat Protoc 13 (5): 1006-1019.
Skene, P. J. and S. Henikoff (2017). “An efficient targeted nuclease strategy for high-resolution mapping of DNA binding sites.” Elife 6.
Smallwood, S. A., H. J. Lee, C. Angermueller, F. Krueger, H. Saadeh, J. Peat, S. R. Andrews, O. Stegle, W. Reik and G. Kelsey (2014). “Single-cell genome-wide bisulfite sequencing for assessing epigenetic heterogeneity.” Nat Methods 11 (8): 817-820.
Stuart, T. and R. Satija (2019). “Integrative single-cell analysis.” Nat Rev Genet 20 (5): 257-272.
Vandereyken, K., Sifrim, A., Thienpont, B. et al. Methods and applications for single-cell and spatial multi-omics. Nat Rev Genet 24, 494-515 (2023).
Vegh, P. and M. Haniffa (2018). “The impact of single-cell RNA sequencing on understanding the functional organization of the immune system.” Brief Funct Genomics 17 (4): 265-272.
Wang, Q., H. Xiong, S. Ai, X. Yu, Y. Liu, J. Zhang and A. He (2019). “CoBATCH for High-Throughput Single-Cell Epigenomic Profiling.” Mol Cell 76 (1): 206-216 e207.
Williams, C. G., Lee, H. J., Asatsuma, T. et al. An introduction to spatial transcriptomics for biomedical research. Genome Med 14, 68 (2022); doi: 10.1186/s13073-022-01075-1.
Wu, S. J., S. N. Furlan, A. B. Mihalas, H. S. Kaya-Okur, A. H. Feroze, S. N. Emerson, Y. Zheng, K. Carson, P. J. Cimino, C. D. Keene, J. F. Sarthy, R. Gottardo, K. Ahmad, S. Henikoff and A. P. Patel (2021). “Single-cell CUT&Tag analysis of chromatin modifications in differentiation and tumor progression.” Nat Biotechnol 39 (7): 819-824.
Zhang, B., A. Srivastava, E. Mimitou, T. Stuart, I. Raimondi, Y. Hao, P. Smibert and R. Satija (2021). “Characterizing cellular heterogeneity in chromatin state with scCUT&amp;Tag-pro.” bioRxiv: 2021.2009.2013.460120.
Zhu, C., Y. Zhang, Y. E. Li, J. Lucero, M. M. Behrens and B. Ren (2021). “Joint profiling of histone modifications and transcriptome in single cells from mouse brain.” Nat Methods 18 (3): 283-292.
Zilionis, R., C. Engblom, C. Pfirschke, V. Savova, D. Zemmour, H. D. Saatcioglu, I. Krishnan, G. Maroni, C. V. Meyerovitz, C. M. Kerwin, S. Choi, W. G. Richards, A. De Rienzo, D. G. Tenen, R. Bueno, E. Levantini, M. J. Pittet and A. M. Klein (2019). “Single-Cell Transcriptomics of Human and Mouse Lung Cancers Reveals Conserved Myeloid Populations across Individuals and Species.” Immunity 50 (5): 1317-1334 e1310.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

1. A method for developing a high efficiency antibody or antibody fragment that targets a histone post-translational modification (PTM) or DNA modification, comprising:
measuring binding specificity of antibody or antibody fragment candidate clones to a recombinant nucleosome substrate comprising the PTM or the DNA modification, thereby identifying a first set of antibodies targeting the histone PTM or the DNA modification; and
validating antibodies or antibody fragments from the first set of antibodies by performing a genomic mapping assay, wherein the first set of antibodies are evaluated for both binding specificity and efficiency, thereby identifying the high efficiency antibody or antibody fragment that targets the histone PTM or DNA modification.

2. The method of claim 1, wherein the method comprises identifying more than one high efficiency antibody or antibody fragment, wherein each high efficiency antibody or antibody fragment targets a different histone PTM and/or DNA modification and the recombinant nucleosome substrate comprises each of the PTMs and/or DNA modifications.

3. The method of claim 1, wherein the validating step is performed with recombinant nucleosomes substrates carrying the histone PTMs or DNA modifications.

4. The method of claim 1, wherein measuring binding specificity comprises performing enzyme-linked immunosorbent assay (ELISA) or performing a multiplex assay, optionally a Luminex assay.

5. (canceled)

6. The method of claim 1, wherein the genomic mapping assay is ChIP-seq, CUT&RUN, or CUT&Tag.

7. The method of claim 6, wherein the genomic mapping assay comprises nucleosome spike-in controls that comprise on-target and off-target epitopes.

8. The method of claim 6, wherein the genomic mapping assay comprises evaluating antibody or antibody fragment binding efficiency at a range of cell inputs.

9. The method of claim 6, wherein the identified high efficiency antibody or antibody fragment generates substantially similar data at 500,000 cells and at fewer than 100,000, 50,000 25,000 10,000, 5000, 1000, 100, or 10 cells.

10. The method of claim 9, wherein data quality is determined using Fragment of Reads in Peaks (FRiP) score or replicate analysis (i.e., Pearson correlation).

11. The method of claim 1, wherein the antibody or antibody fragment is a polyclonal antibody or antibody fragment, monoclonal antibody or antibody fragment, recombinant polyclonal antibody or antibody fragment, recombinant monoclonal antibody or antibody fragment, or nanobody.

12. A method for developing a high efficiency antibody or antibody fragment that targets a chromatin binding protein, comprising:
measuring binding specificity of antibody or antibody fragment candidate clones to a nucleosome substrate comprising an engineered epitope, the epitope found on the target chromatin binding protein, thereby identifying a first set of antibodies targeting the chromatin binding protein; and
validating antibodies or antibody fragments from the first set of antibodies by performing a genomic mapping assay, wherein the first set of antibodies are evaluated for both binding specificity and efficiency, thereby identifying the high efficiency antibody or antibody fragment that targets the chromatin binding protein.

13. The method of claim 12, wherein each step is performed using recombinant nucleosome substrates.

14. The method of claim 12, wherein at least one step is performed using recombinant nucleosome substrates.

15. The method of claim 12, wherein measuring binding specificity comprises performing ELISA or performing a multiplex assay, optionally a Luminex assay.

16. (canceled)

17. The method of claim 12, wherein the genomic mapping assay is ChIP-seq, CUT&RUN, or CUT&Tag.

18. The method of claim 12, wherein the genomic mapping assay comprises nucleosome spike-in controls that comprise on-target and off-target epitopes.

19. The method of claim 17, wherein the genomic mapping assay comprises evaluating antibody or antibody fragment binding efficiency at a range of cell inputs.

20. The method of claim 17, wherein the identified high efficiency antibody or antibody fragment generates substantially similar data at 500,000 cells and at fewer than 100,000, 50,000 25,000 10,000, 5000, 1000, 100, or 10 cells.

21. The method of claim 20, wherein data quality is determined using Fragment of Reads in Peaks (FRiP) score or replicate analysis (i.e., Pearson correlation).

22. The method of claim 12, wherein the antibody or antibody fragment is a polyclonal antibody or antibody fragment, monoclonal antibody or antibody fragment, recombinant polyclonal antibody or antibody fragment, recombinant monoclonal antibody or antibody fragment, or nanobody.

23-27. (canceled)
